(19) United States
(12) Patent Application Publication
(10) Pub. No.: US 2024/0325719 A1
(43) Pub. Date: Oct. 3, 2024

(54) PERCUTANEOUS CIRCULATORY SUPPORT DEVICE INCLUDING PROXIMAL GUIDEWIRE LUMEN

(71) Applicant: Boston Scientific Scimed, Inc., Maple Grove, MN (US)

(72) Inventor: Corey N. Meyer, Zimmerman, MN (US)

(73) Assignee: Boston Scientific Scimed, Inc., Maple Grove, MN (US)

(21) Appl. No.: 18/616,827

(22) Filed: Mar. 26, 2024

Related U.S. Application Data
(60) Provisional application No. 63/455,930, filed on Mar. 30, 2023.

(51) Int. Cl. A61M 60/17; A61M 60/178; A61M 60/216; A61M 60/411; A61M 60/81

(52) U.S. Cl. CPC A61M 60/17; A61M 60/178; A61M 60/216; A61M 60/411; A61M 60/81; A61M 2210/125

(57) ABSTRACT

A percutaneous circulatory support device includes an impeller housing configured to receive a guidewire during delivery of the percutaneous circulatory support device to a patient. A catheter is coupled to a proximal end portion of the impeller housing, the catheter including a lumen being configured to receive the guidewire during delivery of the percutaneous circulatory support device to the patient. An impeller is rotatable in the impeller housing after delivery of the percutaneous circulatory support device to the patient to cause blood to flow through the percutaneous circulatory support device.

### CROSS-REFERENCE TO RELATED APPLICATIONS

This application claims the benefit of U.S. Provisional Patent Application Ser. No. 63/455,930, filed Mar. 30, 2023, which is incorporated herein by reference.

### TECHNICAL FIELD

The present disclosure relates to percutaneous circulatory support devices. More specifically, the present disclosure relates to percutaneous circulatory support devices delivered using guidewires.

### BACKGROUND

Percutaneous circulatory support devices (which are also referred to, interchangeably, as “blood pumps”) can provide transient support for up to approximately several weeks in patients with compromised heart function or cardiac output. FIG. 1 depicts a blood pump 100 according to the prior art. The blood pump 100 is delivered percutaneously by advancing along a guidewire 102 positioned within the patient, the guidewire 102 specifically extending though the aorta 10, the aortic valve 12, and into the left ventricle 14 of the heart 16. The blood pump 100 receives the guidewire 102 such that the guidewire 102 extends through a cannula 104 of the pump 100, through an impeller housing 106 of the pump 100, and out of an outlet 108 of the impeller housing 106. As illustrated, when the pump 100 passes through and is disposed distally from the aortic arch 18, the guidewire 102 extends from the outlet 108 at a relatively steep angle. The guidewire 102 thereby may be subjected to relatively high contact forces at the outlet 108 of the impeller housing 106, which can damage an outer coating of the guidewire 102. Accordingly, improved devices would be beneficial.

### SUMMARY

In an Example 1, a percutaneous circulatory support device includes an impeller housing configured to receive a guidewire during delivery of the percutaneous circulatory support device to a patient. A catheter is coupled to a proximal end portion of the impeller housing, the catheter including a lumen being configured to receive the guidewire during delivery of the percutaneous circulatory support device to the patient. An impeller is rotatable in the impeller housing after delivery of the percutaneous circulatory support device to the patient to cause blood to flow through the percutaneous circulatory support device.

In an Example 2, the percutaneous circulatory support device of Example 1, wherein the impeller housing includes an outlet, the impeller being rotatable in the impeller housing to cause blood to flow through the percutaneous circulatory support device and out of the outlet, and the outlet permitting the guidewire to extend therethrough during delivery of the percutaneous circulatory support device to the patient.

In an Example 3, the percutaneous circulatory support device of any of Examples 1-2, wherein the lumen is an auxiliary lumen, the catheter further includes a primary lumen, and further including a cable disposed in the primary lumen, the cable being operatively coupled to the impeller.

In an Example 4, the percutaneous circulatory support device of Example 3, wherein the catheter includes a first wall and a second wall coupled to the first wall, the first wall defining the primary lumen and the second wall defining the auxiliary lumen.

In an Example 5, the percutaneous circulatory support device of Example 4, further comprising a connector coupling the auxiliary lumen to the primary lumen.

In an Example 6, the percutaneous circulatory support device of Example 3, wherein the catheter includes an outer circumferential surface, and the primary lumen and the auxiliary lumen are disposed radially inwardly from the outer circumferential surface.

In an Example 7, the percutaneous circulatory support device of any of Examples 1-6, wherein the impeller housing further includes a distal end portion opposite the proximal end portion, and further including a cannula coupled to the distal end portion of the impeller housing, the cannula being configured to receive the guidewire during delivery of the percutaneous circulatory support device to the patient.

In an Example 8, the percutaneous circulatory support device of any of Examples 1-6, wherein the lumen has a length in a range of 1 in. to 12 in.

In an Example 9, a percutaneous circulatory support device includes a catheter including a lumen. A housing is coupled to the catheter and including an inlet and an outlet, the housing being configured to receive a guidewire during delivery of the percutaneous circulatory support device to a patient such that the guidewire extends out of the outlet and through the lumen of the catheter. An impeller is disposed distally relative to the catheter and coupled to the housing, the impeller being rotatable to cause blood to flow into the inlet, through the housing, and out of the outlet after delivery of the percutaneous circulatory support device to the patient.

In an Example 10, the percutaneous circulatory support device of Example 9, wherein the lumen is an auxiliary lumen, the catheter further includes a primary lumen, and further including a cable disposed in the primary lumen, the cable being operatively coupled to the impeller.

In an Example 11, the percutaneous circulatory support device of Example 10, wherein the catheter includes a first wall and a second wall coupled to the first wall, the first wall defining the primary lumen and the second wall defining the auxiliary lumen.

In an Example 12, the percutaneous circulatory support device of Example 11, further comprising a connector coupling the auxiliary lumen to the primary lumen.

In an Example 13, the percutaneous circulatory support device of Example 10, wherein the catheter includes an outer circumferential surface, and the primary lumen and the auxiliary lumen are disposed radially inwardly from the outer circumferential surface.

In an Example 14, the percutaneous circulatory support device of any of Examples 9-13, further including a cannula disposed distally relative to the impeller, wherein the percutaneous circulatory support device is configured to receive the guidewire during delivery to the patient such that the guidewire extends through the cannula, out of the outlet, and through the lumen of the catheter.

In an Example 15, the percutaneous circulatory support device of any of Examples 9-14, wherein the lumen has a length in a range of 1 in. to 12 in.

In an Example 16, a percutaneous circulatory support device includes an impeller housing including a proximal end portion, and the impeller housing being configured to receive a guidewire during delivery of the percutaneous circulatory support device to a patient. An impeller is rotatable in the impeller housing to cause blood to flow through the percutancous circulatory support device. A catheter is coupled to the proximal end portion of the impeller housing, the catheter including a lumen being configured to receive the guidewire during delivery of the percutaneous circulatory support device to the patient.

In an Example 17, the percutaneous circulatory support device of Example 16, wherein the impeller housing includes an outlet, the impeller being rotatable in the impeller housing to cause blood to flow through the percutaneous circulatory support device and out of the outlet. The outlet permits the guidewire to extend therethrough during delivery of the percutaneous circulatory support device to the patient.

In an Example 18, the percutaneous circulatory support device of Example 16, wherein the lumen is an auxiliary lumen, the catheter further includes a primary lumen, and further including a cable disposed in the primary lumen, the cable being operatively coupled to the impeller.

In an Example 19, the percutaneous circulatory support device of Example 18, wherein the catheter includes a first wall and a second wall coupled to the first wall, and the first wall defines the primary lumen and the second wall defines the auxiliary lumen.

In an Example 20, the percutaneous circulatory support device of Example 18, wherein the catheter includes an outer circumferential surface, and the primary lumen and the auxiliary lumen are disposed radially inwardly from the outer circumferential surface.

In an Example 21, the percutancous circulatory support device of Example 16, wherein the impeller housing further includes a distal end portion opposite the proximal end portion, and further including a cannula coupled to the distal end portion of the impeller housing, the cannula being configured to receive the guidewire.

In an Example 22, the percutaneous circulatory support device of Example 16, wherein the lumen has a length in a range of 1 in. to 12 in.

In an Example 23, a percutaneous circulatory support device includes an inlet, an outlet, and an impeller being rotatable to cause blood to flow into the inlet, through the percutaneous circulatory support device, and out of the outlet. A catheter is disposed proximally relative to the impeller, and the catheter includes a lumen. The percutaneous circulatory support device is configured to receive a guidewire during delivery to a patient such that the guidewire extends out of the outlet and through the lumen of the catheter.

In an Example 24, the percutaneous circulatory support device of Example 23, wherein the lumen is an auxiliary lumen, the catheter further includes a primary lumen, and further including a cable disposed in the primary lumen, the cable being operatively coupled to the impeller.

In an Example 25, the percutaneous circulatory support device of Example 24, wherein the catheter includes a first wall and a second wall coupled to the first wall, the first wall defining the primary lumen and the second wall defining the auxiliary lumen.

In an Example 26, the percutaneous circulatory support device of Example 24, wherein the catheter includes an outer circumferential surface, and the primary lumen and the auxiliary lumen are disposed radially inwardly from the outer circumferential surface.

In an Example 27, the percutaneous circulatory support device of Example 23, further including a cannula disposed distally relative to the impeller, wherein the percutaneous circulatory support device is configured to receive the guidewire during delivery to the patient such that the guidewire extends through the cannula, out of the outlet, and through the lumen of the catheter.

In an Example 28, the percutaneous circulatory support device of Example 23, wherein the lumen has a length in a range of 1 in. to 12 in.

In an Example 29, a method of delivering a percutaneous circulatory support device to a patient includes: positioning a guidewire in the patient; inserting the guidewire through a housing of the percutaneous circulatory support device and into a lumen of a catheter of the percutaneous circulatory support device; and advancing the percutaneous circulatory support device along the guidewire.

In an Example 30, the method of Example 29, wherein inserting the guidewire includes inserting the guidewire through the housing and out of an outlet of the housing.

In an Example 31, the method of Example 29, wherein inserting the guidewire includes inserting the guidewire through a cannula of the percutaneous circulatory support device.

In an Example 32, the method of Example 29, wherein the lumen of the catheter is an auxiliary lumen, the catheter further includes a primary lumen, and the percutaneous circulatory support device further includes a cable disposed in the primary lumen, the cable being operatively coupled to an impeller of the percutaneous circulatory support device.

In an Example 33, the method of Example 32, wherein the catheter includes a first wall and a second wall coupled to the first wall, the first wall defining the primary lumen and the second wall defining the auxiliary lumen.

In an Example 34, the method of Example 32, wherein the catheter includes an outer circumferential surface, and the primary lumen and the auxiliary lumen are disposed radially inwardly from the outer circumferential surface.

In an Example 35, the method of Example 29, wherein the lumen has a length in a range of 1 in. to 12 in.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a partial side view of an illustrative percutaneous circulatory support device positioned in the aorta and the left ventricle of a patient, in accordance with the prior art.

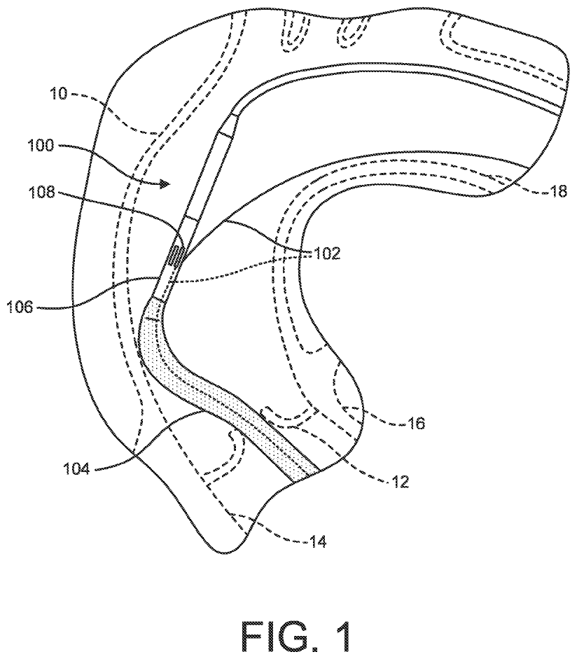

FIG. 2 is a partial side view of an illustrative percutaneous circulatory support device positioned in the aorta and the left ventricle of a patient, in accordance with embodiments of the subject matter disclosed herein.

FIG. 3 is another partial side view of the percutaneous circulatory support device of FIG. 2.

FIG. 4 is a partial side sectional view of a proximal portion of the percutaneous circulatory support device of FIG. 2.

FIG. 5 is a partial side sectional view of a proximal portion of another illustrative percutaneous circulatory support device, in accordance with embodiments of the subject matter disclosed herein.

FIG. 6 is a diagram of an illustrative method of delivering a percutaneous circulatory support device in a patient, in accordance with embodiments of the subject matter disclosed herein.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

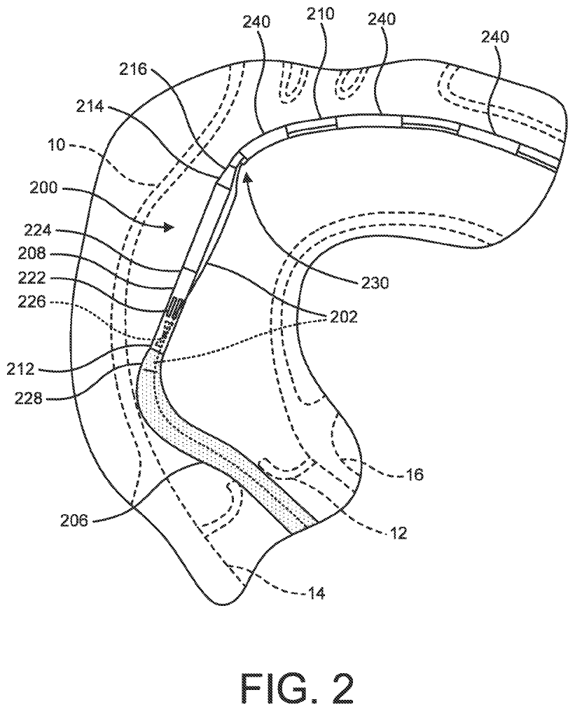

FIG. 2 depicts a portion of an illustrative percutaneous mechanical circulatory support device 200 (also referred to herein, interchangeably, as a “blood pump”), and its relative position in a human aorta 10 and heart 16, more specifically the left ventricle 14, in accordance with embodiments of the subject matter disclosed herein. The blood pump 200 is delivered percutaneously by using a guidewire 202 and passing through the aorta 10, and positioned in the heart 16 with respect to the left ventricle 14 and the aortic valve 12, as shown in FIG. 2. In contrast to the blood pump 100, the blood pump 200 inhibits the guidewire 202 from extending from the pump 200 at a relatively steep angle, and thereby inhibits relatively high contact forces and potential damage to the guidewire 202. These aspects are described in further detail below.

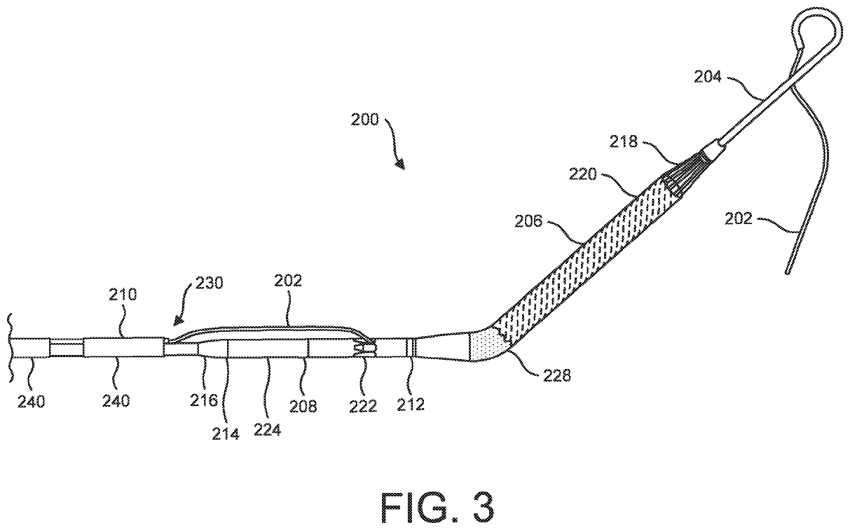

With continued reference to FIG. 2 and additional reference to FIG. 3, the blood pump 200 generally includes a distal extension 204 (FIG. 3), a cannula 206, an impeller portion 208 and a proximal catheter 210. The cannula 206 couples to a distal end portion 212 of the impeller portion 208, and the catheter 210 couples to a proximal end portion 214 of the impeller portion 208, for example, via a tapering connector 216. The cannula 206 and the catheter 210 may have flexible constructions to facilitate delivery of the blood pump 200. The cannula 206 includes one or more blood inlets 218 (FIG. 3) located on a distal portion 220 thereof, and one or more blood outlets 222 are located on an impeller housing 224 of the impeller portion 208. The housing 224 carries an impeller 226 (FIG. 2), and the impeller 226 rotates relative to the housing 224 to cause blood to flow into the inlets 218, through the housing 224, and out of the outlets 222. Upon delivery and as shown in FIG. 2, the blood pump 200 is positioned within the heart 16 such that the inlets 218 (FIG. 3) are positioned in the left ventricle 14 and the outlets 222 are positioned in the aorta 10. As a result, rotation of the impeller 226 relative to the housing 224 causes blood to flow from the left ventricle 14, through the cannula 206 and the impeller portion 208, and into the aorta 10.

With continued reference to FIGS. 2 and 3, the cannula 206 includes a preformed curve (as used herein, the term “preformed” and variations thereof meaning that a feature is present prior to entering the vasculature of a patient). More specifically, the cannula 206 includes a bent longitudinal axis that provides a preformed curve 228. Although not specifically illustrated, in some embodiments the catheter 210 also includes one or more preformed curves.

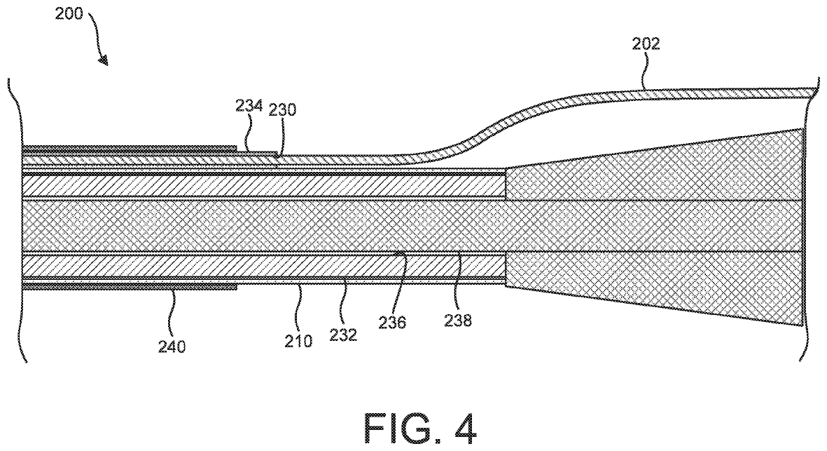

With continued reference to FIGS. 2 and 3 and additional reference to FIG. 4, during delivery the pump 200 receives and is advanceable along the guidewire 202. More specifically, a distal end portion 212 of the guidewire 202 is disposed distally from the distal extension 204. The guidewire 202 extends into and through the distal extension 204, through the cannula 206, through the impeller housing 224 and out of the outlet 222. In some embodiments and as discussed below, a guidewire insertion aide (not shown) may be incorporated into the pump 200 to facilitate the passage of the guidewire 202 through the pump 200. In contrast to the blood pump 100, the guidewire 202 also extends into and through the proximal catheter 210. More specifically, the guidewire 202 extends into and through an auxiliary or guidewire lumen 230 of the catheter 210. By receiving the guidewire 202 in this manner, the blood pump 200 inhibits the guidewire 202 from extending from the outlet 222 at a relatively steep angle, and thereby inhibits relatively high contact forces (that is, frictional contact) from occurring between the housing 224 (specifically, at the outlets 222) and the guidewire 202 and thus reducing potential damage to the guidewire 202 from such forces. Additionally, by receiving the guidewire 202 in this manner, the pump 200 inhibits the guidewire 202 from wrapping around distal portions of the pump 200.

The auxiliary lumen 230 has a length sufficient for maintaining the guidewire 202 in close proximity to the blood pump 200, and in particular to the catheter 210, in the aortic arch of the patient. In addition, the auxiliary lumen 230 may terminate in the descending aorta. For example, the auxiliary lumen 230 may have a length in a range of 1 inch (herein, “in.”) to 12 in. As another example, the auxiliary lumen 230 may have a length that is substantially equal to that of the catheter 210. As yet another example, the auxiliary lumen 230 may be offset from the housing 224 by about 0.8 in. and have a length of about 0.25 in. In some embodiments, the auxiliary lumen 230 has a diameter sufficient for receiving both the guidewire 202 and a guidewire insertion aide (not shown), such as any of the guidewire insertion aides contemplated in U.S. Application No. 63/384,333, filed Nov. 18, 2022, the disclosure of which is hereby incorporated by reference. For example, the auxiliary lumen 230 may have a diameter in range from a minimum diameter to a maximum diameter, the minimum diameter being sufficient to receive a guidewire, and the maximum diameter being sufficient to receive a guidewire insertion aide. More specifically, the auxiliary lumen 230 may have a diameter in a range of 0.018 in. to 0.042 in.

With reference to FIG. 4, the catheter 210 illustratively includes a first tubular wall 232 and a second tubular wall 234. The first wall 232 defines a primary lumen 236 and the second wall 234 defines the auxiliary lumen 230. The primary lumen 236 carries, for example, a cable 238 that operatively couples to the impeller 226 (shown elsewhere). More specifically, the cable 238 powers a motor (not shown) that indirectly or directly drives the impeller 226. Other components, such as cables coupled to sensors, for example pressure sensors, may also pass through the primary lumen 236. The first wall 232 and the second wall 234 may be coupled in various manners, such as bonding, welding, polymer reflow, and/or one or more heat shrink connectors 240. As described herein, the auxiliary lumen 230 carries the guidewire 202, at least during delivery of the blood pump 200 into the aorta 10 and heart 16.

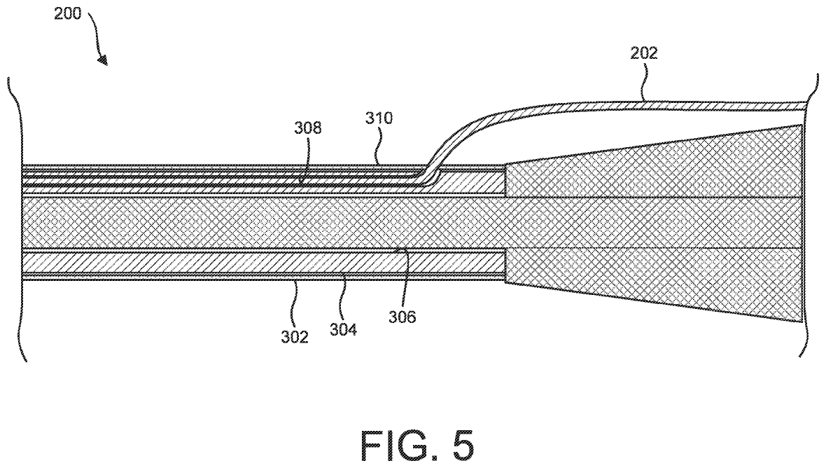

Blood pumps in accordance with embodiments of the subject matter disclosed herein may include catheters with different structures. For example, FIG. 5 illustrates a proximal portion of another illustrative percutaneous circulatory support device 300, in accordance with embodiments of the subject matter disclosed herein. The blood pump 300 is generally similar to the blood pump 200 described hereinabove, except that that the catheter 302 includes a different structure. More specifically, the catheter 302 includes a tubular wall 304, and the primary lumen 306 is disposed radially inwardly from the tubular wall 304, and the auxiliary lumen 308 is disposed within the tubular wall 304. Stated another way, the catheter 302 comprises an outer circumferential surface 310, and the primary lumen 306 and the auxiliary lumen 308 are disposed radially inwardly from the outer circumferential surface 310. As described above with respect to the blood pump 200, the primary lumen 306 may carry one or more cables that operatively couple to a motor and/or one or more sensors, and the auxiliary lumen 308 is configured to carry a guidewire.

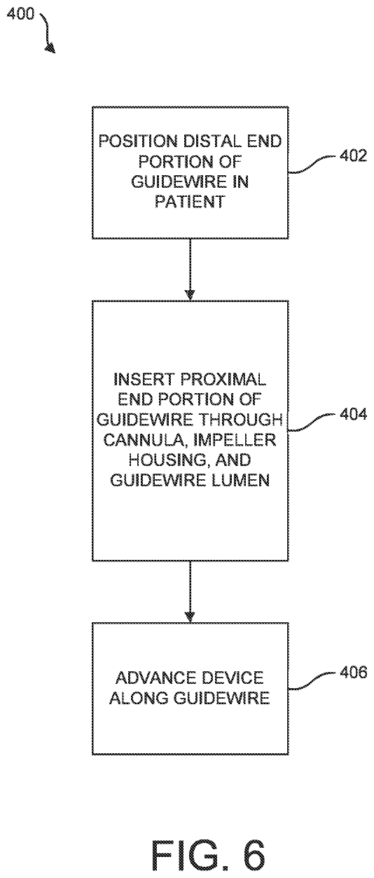

FIG. 6 illustrates a diagram of an exemplary method 400 of delivering a percutaneous circulatory support device in a patient, in accordance with embodiments of the subject matter disclosed herein. The following description of the method 400 only refers to the blood pump 200 and its features as illustrated in FIGS. 2-4, although it is understood that any of the blood pumps contemplated herein may be delivered in a similar manner. At block 402, the method begins by positioning the guidewire 202 in the patient. More specifically, the distal end portion 212 of the guidewire 202 is percutaneously inserted in the patient, advanced through the aorta 10, and through the aortic valve 12, and into the left ventricle 14. A proximal end portion of the guidewire 202 (not shown) remains positioned outside of the patient. At block 404, the method continues by inserting the proximal end portion of the guidewire 202 through the distal extension 204, through the cannula 206, through the impeller housing 224, out of the outlet 222, and through the guidewire lumen 230 of the catheter 210. At block 406, the blood pump 200 is advanced distally along the guidewire 202 until the blood pump 200 is properly positioned in the patient. More specifically, the blood pump 200 is positioned in the aorta 10, across the aortic valve 12, and in the left ventricle 14 as shown in FIG. 2.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A percutaneous circulatory support device, comprising:
an impeller housing comprising a proximal end portion, and the impeller housing being configured to receive a guidewire during delivery of the percutaneous circulatory support device to a patient;
an impeller being rotatable in the impeller housing to cause blood to flow through the percutaneous circulatory support device; and
a catheter coupled to the proximal end portion of the impeller housing, the catheter comprising a lumen being configured to receive the guidewire during delivery of the percutaneous circulatory support device to the patient.

2. The percutaneous circulatory support device of claim 1, wherein the impeller housing comprises an outlet, the impeller being rotatable in the impeller housing to cause blood to flow through the percutaneous circulatory support device and out of the outlet, and the outlet permitting the guidewire to extend therethrough during delivery of the percutaneous circulatory support device to the patient.

3. The percutaneous circulatory support device of claim 1, wherein the lumen is an auxiliary lumen, the catheter further comprises a primary lumen, and further comprising a cable disposed in the primary lumen, the cable being operatively coupled to the impeller.

4. The percutaneous circulatory support device of claim 3, wherein the catheter comprises a first wall and a second wall coupled to the first wall, the first wall defining the primary lumen and the second wall defining the auxiliary lumen.

5. The percutaneous circulatory support device of claim 4, wherein the catheter comprises an outer circumferential surface, and the primary lumen and the auxiliary lumen are disposed radially inwardly from the outer circumferential surface.

6. The percutaneous circulatory support device of claim 1, wherein the impeller housing further comprises a distal end portion opposite the proximal end portion, and further comprising a cannula coupled to the distal end portion of the impeller housing, the cannula being configured to receive the guidewire.

7. The percutaneous circulatory support device of claim 1, wherein the lumen has a length in a range of 1 in. to 12 in.

8. A percutaneous circulatory support device, comprising:
an inlet;
an outlet;
an impeller being rotatable to cause blood to flow into the inlet, through the percutaneous circulatory support device, and out of the outlet; and
a catheter disposed proximally relative to the impeller, the catheter comprising a lumen;
wherein the percutaneous circulatory support device is configured to receive a guidewire during delivery to a patient such that the guidewire extends out of the outlet and through the lumen of the catheter.

9. The percutaneous circulatory support device of claim 8, wherein the lumen is an auxiliary lumen, the catheter further comprises a primary lumen, and further comprising a cable disposed in the primary lumen, the cable being operatively coupled to the impeller.

10. The percutaneous circulatory support device of claim 9, wherein the catheter comprises a first wall and a second wall coupled to the first wall, the first wall defining the primary lumen and the second wall defining the auxiliary lumen.

11. The percutaneous circulatory support device of claim 9, wherein the catheter comprises an outer circumferential surface, and the primary lumen and the auxiliary lumen are disposed radially inwardly from the outer circumferential surface.

12. The percutaneous circulatory support device of claim 8, further comprising a cannula disposed distally relative to the impeller, wherein the percutaneous circulatory support device is configured to receive the guidewire during delivery to the patient such that the guidewire extends through the cannula, out of the outlet, and through the lumen of the catheter.

13. The percutaneous circulatory support device of claim 8, wherein the lumen has a length in a range of 1 in. to 12 in.

14. A method of delivering a percutaneous circulatory support device to a patient, the method comprising:
positioning a guidewire in the patient;
inserting the guidewire through a housing of the percutaneous circulatory support device and into a lumen of a catheter of the percutaneous circulatory support device; and
advancing the percutaneous circulatory support device along the guidewire.

15. The method of claim 14, wherein inserting the guidewire comprises inserting the guidewire through the housing and out of an outlet of the housing.

16. The method of claim 14, wherein inserting the guidewire comprises inserting the guidewire through a cannula of the percutaneous circulatory support device.

17. The method of claim 14, wherein the lumen of the catheter is an auxiliary lumen, the catheter further comprises a primary lumen, and the percutaneous circulatory support device further comprises a cable disposed in the primary lumen, the cable being operatively coupled to an impeller of the percutaneous circulatory support device.

18. The method of claim 17, wherein the catheter comprises a first wall and a second wall coupled to the first wall, the first wall defining the primary lumen and the second wall defining the auxiliary lumen.

19. The method of claim 17, wherein the catheter comprises an outer circumferential surface, and the primary lumen and the auxiliary lumen are disposed radially inwardly from the outer circumferential surface.

20. The method of claim 14, wherein the lumen has a length in a range of 1 in. to 12 in.
